(19) United States
(12) Patent Application Publication
(10) Pub. No.: US 2006/0264826 A1
(43) Pub. Date: Nov. 23, 2006

(54) Self-destructive safety syringe

(76) Inventor: Chang-Tzu Chen, Taipei (TW)

Correspondence Address: John G. Chupa;Law Offices of John Chupa & Associates, P.C., Suite 50, 28535 Orchard Lake Road, Farmington Hills, MI, 48334, US

(21) Appl. No.: 11/182,979

(22) Filed: Jul. 15, 2005

(30) Foreign Application Priority Data
May 19, 2005 (TW) 094208118

(51) Int. Cl. A61M 5/00

(52) U.S. Cl. 604/110

(57) ABSTRACT

A self-destructive safety syringe having a thin breaking wall portion at the front wall of the barrel thereof that is breakable with the plunger of the syringe when the user forcing a barbed front tip of the plunger into engagement with the tubular front neck of the barrel after the service of the syringe, for enabling the plunger to carry the tubular front neck of the barrel and the attached needle assembly to the inside of the barrel after the service of the syringe to prevent contamination.

### BACKGROUND OF THE INVENTION

1. Field of the Invention

The present invention relates to a safety hypodermic syringe and more particularly, to a self-destructive safety syringe that can be destructed to keep the needle assembly firmly received inside the barrel after the service of the syringe.

2. Description of the Related Art

A hypodermic syringe is a medical instrument used to inject or withdraw fluids. However, improper use of a hypodermic syringe or improper disposal of a used hypodermic syringe may cause a contamination. In order to prevent contamination after use, safety hypodermic syringes are developed. A safety hypodermic syringe is known comprising a needle assembly, a medicine barrel, a plunger, and an outer barrel. The needle assembly is fastened to the front side of the medicine barrel. The plunger is axially movably coupled to the medicine barrel and mounted with the medicine barrel inside the outer barrel. After the service of the hypodermic syringe, the plunger is pulled backwards to carry the needle cannula or the needle assembly backwards to the inside of the medicine barrel. This design of safety hypodermic syringe has a complicated engagement structure for connection between the plunger and the needle assembly or needle cannula.

The conventional safety hypodermic syringes are functional. However, their complicated structure results in a high manufacturing cost.

### SUMMARY OF THE INVENTION

The present invention has been accomplished under the circumstances in view. It is the main object of the present invention to provide a syringe, which is self-destructive, preventing the possibility of a reuse. It is another object of the present invention to provide a self-destructive safety syringe, which is easy and inexpensive to manufacture. It is still another object of the present invention to provide a self-destructive safety syringe, which keeps the needle assembly firmly secured to the inside of the barrel, preventing possible contamination. To achieve these and other objects of the present invention, the self-destructive safety syringe comprises a barrel, the barrel having a tubular front neck forwardly extending from a front wall thereof, a needle assembly fastened to the tubular front neck of the barrel, a plunger axially movably inserted into the barrel, and a rubber stopper mounted on the plunger and peripherally maintained in close contact with an inside wall of the barrel and axially movable with the plunger relative to the barrel, wherein the front wall of the barrel has at least one thin breaking wall portion through which the front wall can be broken with the plunger for enabling the tubular front neck and the needle assembly to be pulled backwards into the inside of the barrel by the plunger after the service of the self-destructive safety syringe; the plunger has a barbed tip protruding over a front side of the rubber stopper for hooking the tubular front neck to pull the tubular front neck and the needle assembly backwards into the inside of the barrel when the at least one thin breaking wall portion is broken.

### BRIEF DESCRIPTION OF THE DRAWINGS

FIG. 1 is an exploded view of a self-destructive safety syringe according to the present invention.

FIG. 2 is an elevational assembly view of the self-destructive safety syringe according to the present invention.

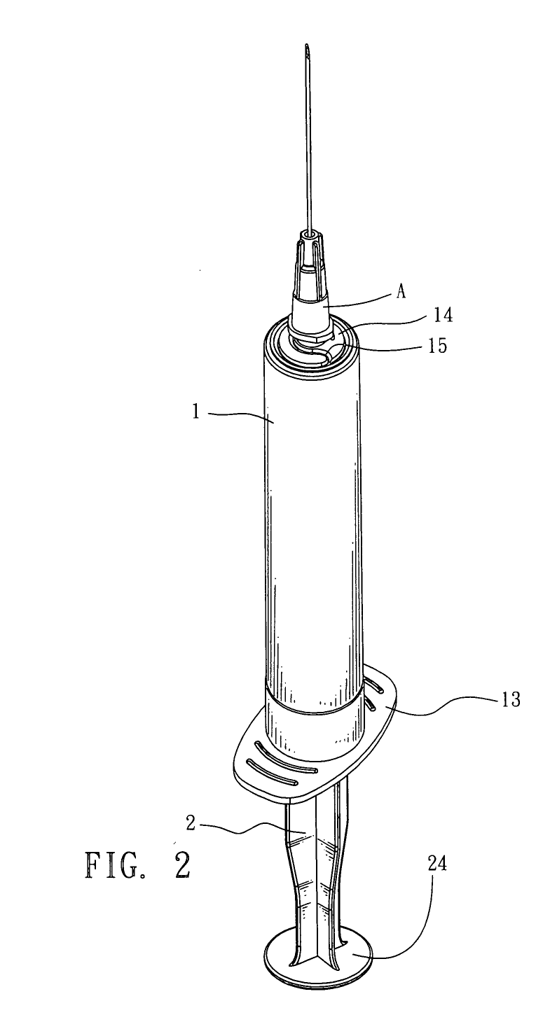

FIG. 3 is a sectional view of the present invention showing a status of the self-destructive safety syringe after the service.

FIG. 4 is a schematic sectional view showing the plunger pushed against the front wall of the barrel after the service of the syringe and the breaking portion of the front wall of the barrel broken according to the present invention.

FIG. 5 is a schematic sectional view showing the broken part of the barrel and the attached needle assembly carried to the inside of the cylindrical body of the barrel according to the present invention.

FIG. 6 corresponds to FIG. 5, showing the broken part of the barrel and the attached needle assembly positioned in the barrel after the service of the syringe.

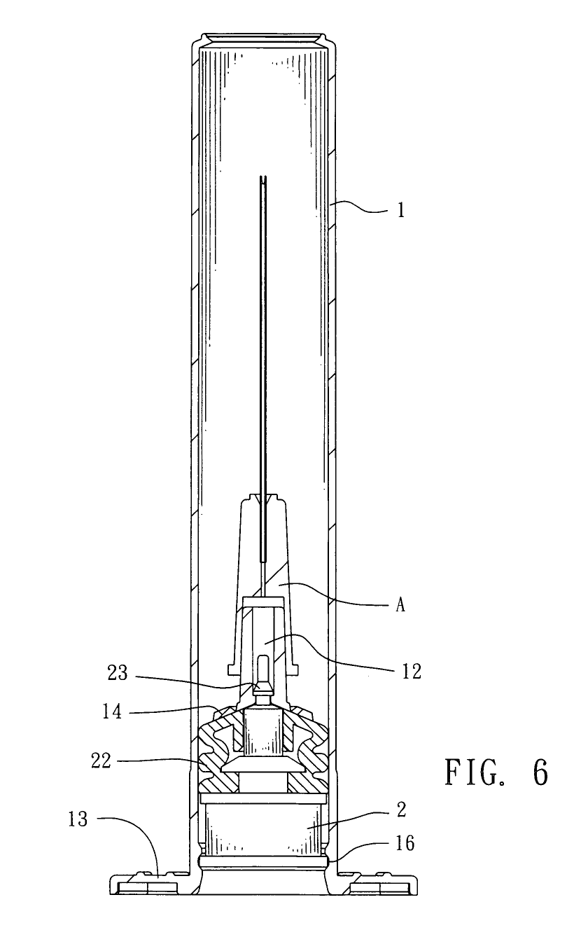

### DETAILED DESCRIPTION OF THE PREFERRED EMBODIMENT

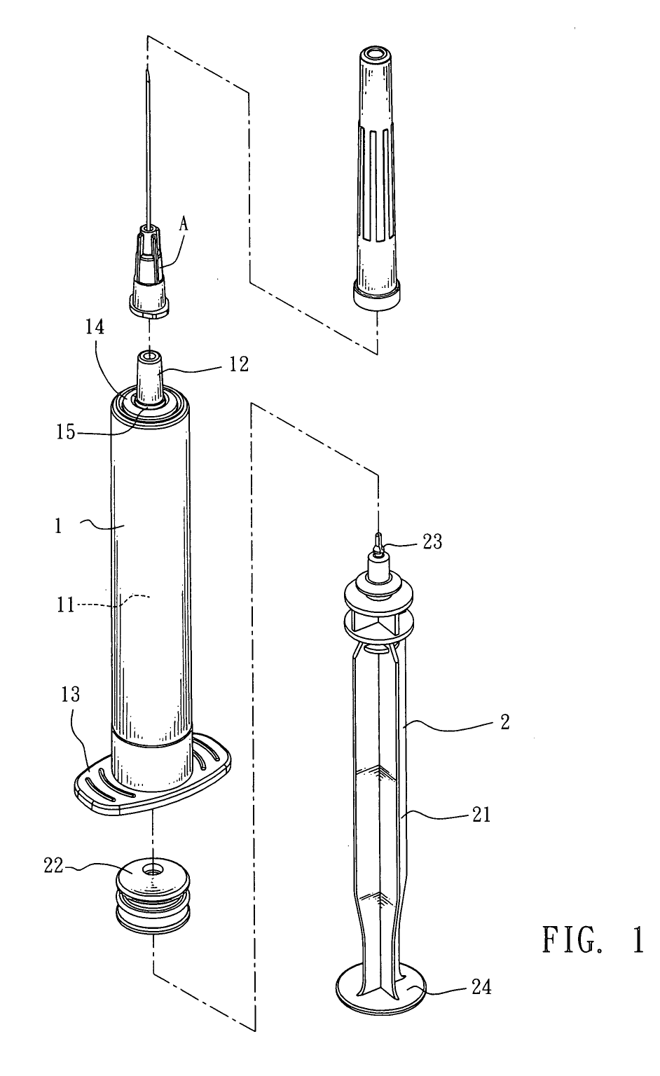

Referring to FIGS. 1˜3, a self-destructive safety syringe in accordance with the present invention is shown comprised of a barrel 1, a plunger 2, a rubber stopper 22, and a needle assembly A.

Similar to the barrel of a conventional syringe, the barrel 1 comprises a cylindrical body 11 adapted to hold a liquid medicine, a tubular neck 12 forwardly and coaxially extending from the front side of the cylindrical body 14, and a finger flange 13 extending around the rear side of the cylindrical body 14. Further, the barrel 1 has a front wall 14 at the front side, a breaking portion 15 formed in the front wall 14, and an inside annular groove 16 extending around the inside wall near the rear side. The tubular neck 12 is formed integral with the front wall 14. The breaking portion 15 is a thin wall portion that can be made in the shape of a straight line, spiral line, or S-line. The breaking portion 15 can easily be broken when the user pushes the plunger 2 with force, allowing the tubular neck 12 and the needle assembly A to be carried backwards into the inside of the cylindrical body 11 with the plunger 2 after the service of the syringe.

The plunger 2 comprises a shank 21, a thumb rest 24 at the rear side of the shank 21, and a barbed tip 23 at the front side. The rubber stopper 22 is fastened to a part of the plunger 2 between the barbed tip 23 and the shank 21, and maintained in close contact with the inside wall of the cylindrical body 11 of the barrel 2. The connection part of the plunger 2 between the shank 21 and the barbed tip 23 for holding the rubber stopper 22 has a breaking portion 25 connected to the shank 21. Through the breaking portion 25, the plunger 2 can easily be broken.

When the user pulls the plunger 2 backwards relative to the barrel 1, a suction force is produced to draw the liquid medicine from the vial (not shown) into the cylindrical body 11 of the barrel 1 through the needle assembly A. When pushing the plunger 2 forwards after insertion of the needle cannula of the needle assembly A into the patient's body, the liquid medicine is forced out of the cylindrical body 11 of the barrel 1 through the needle assembly A into the patient's body.

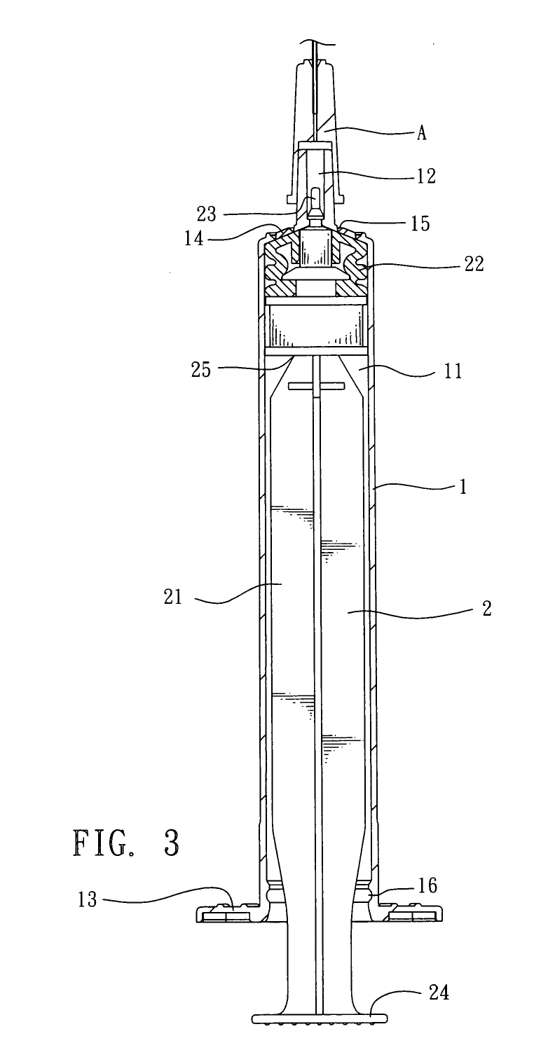

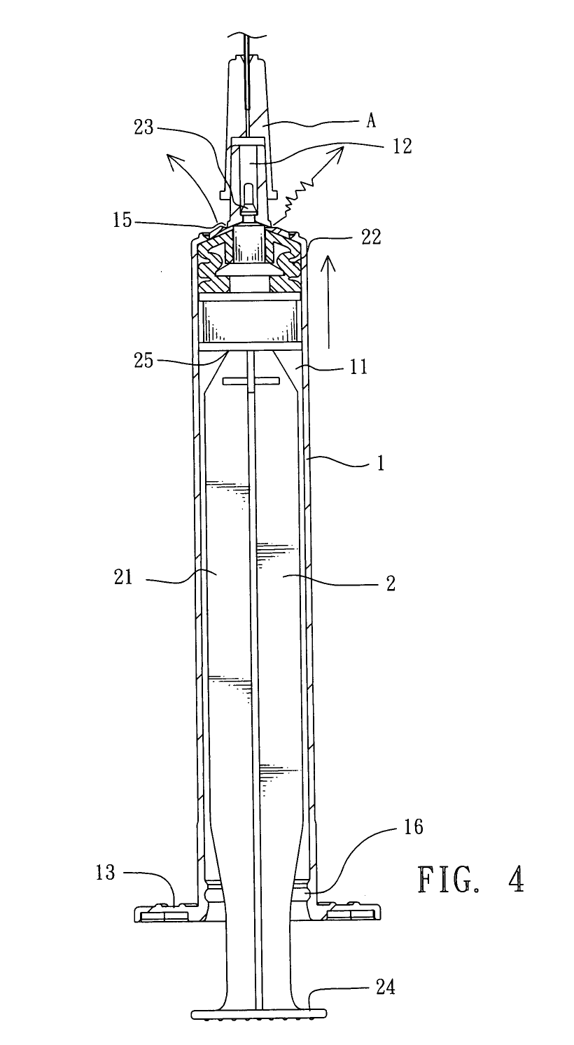

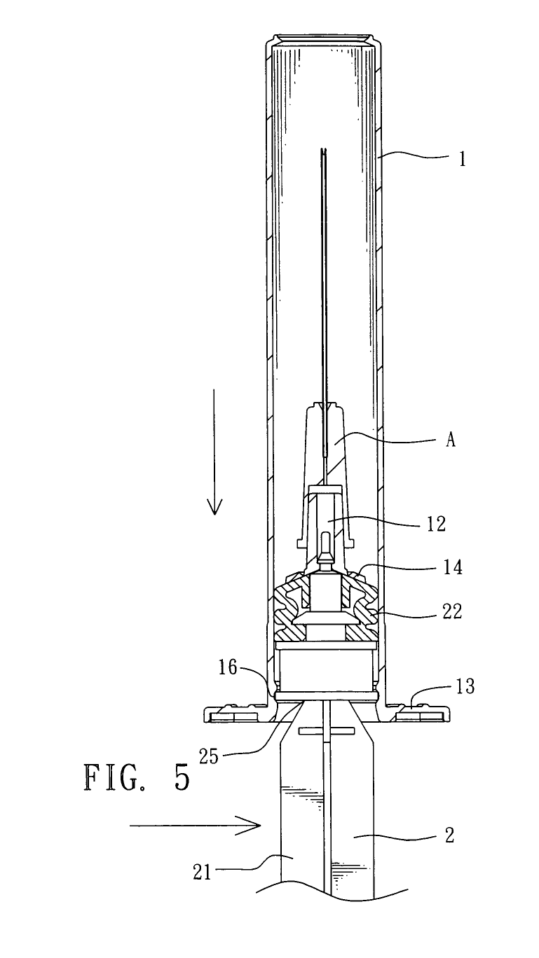

After the service of the syringe (see FIG. 3), push the plunger 2 forwards with force to break the breaking portion 15 and to move the barbed tip 23 into engagement with the tubular front neck 12 (see FIG. 4), and then pull the plunger 2 backwards to carry the broken front wall 14 with the tubular front neck 12 and the needle assembly A into the inside of the cylindrical body 11 of the barrel 1 and to have the connection part of the plunger 2 between the shank 21 and the barbed tip 23 be forced into engagement with the inside annular groove 16 of the barrel 1 (see FIG. 5), and then bias the shank 21 from the breaking portion 25, keeping the broken front part of the plunger 2 with the rubber stopper 22 and the needle assembly A secured to the inside of the cylindrical body 11 of the barrel 1.

A prototype of self-destructive safety syringe has been constructed with the features of FIGS. 1˜6. The self-destructive safety syringe functions smoothly to provide all the features discussed earlier.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A self-destructive safety syringe comprising a barrel, said barrel having a tubular front neck forwardly extending from a front wall thereof, a needle assembly fastened to said tubular front neck of said barrel, a plunger axially movably inserted into said barrel, and a rubber stopper mounted on said plunger and peripherally maintained in close contact with an inside wall of said barrel and axially movable with said plunger relative to said barrel, wherein said front wall of said barrel has at least one thin breaking wall portion through which said front wall can be broken with said plunger for enabling said tubular front neck and said needle assembly to be pulled backwards into the inside of said barrel by said plunger after the service of the self-destructive safety syringe; said plunger has a barbed tip protruding over a front side of said rubber stopper for hooking said tubular front neck to pull said tubular front neck and said needle assembly backwards into the inside of said barrel when said at least one thin breaking wall portion is broken.

2. The self-destructive safety syringe as claimed in claim 1, wherein said barrel has a finger flange at a rear side thereof.

3. The self-destructive safety syringe as claimed in claim 1, wherein said plunger has a thumb rest at a rear side thereof opposite to said barbed tip.

4. The self-destructive safety syringe as claimed in claim 1, wherein said plunger comprises a thin breaking portion disposed at a rear side of said rubber stopper through which said plunger is breakable with force.

5. The self-destructive safety syringe as claimed in claim 1, wherein the number of said at least one thin breaking wall portion of said front wall of said barrel is 2.

6. The self-destructive safety syringe as claimed in claim 1, wherein said at least one thin breaking wall portion of said front wall of said barrel each has a S-shape.

7. The self-destructive safety syringe as claimed in claim 1, wherein said barrel has an inside annular groove disposed near a rear side thereof.
